(19) United States
(12) Patent Application Publication
(10) Pub. No.: US 2013/0261611 A1
(43) Pub. Date: Oct. 3, 2013

(54) Modular Diagnostic Instrument Workstation Architecture and Method

(71) Applicants: Ashish Dhar, Peekskill, NY (US); Thinakaran Kesavan, West Havestraw, NY (US)

(72) Inventors: Ashish Dhar, Peekskill, NY (US); Thinakaran Kesavan, West Havestraw, NY (US)

(73) Assignee: Siemens Healthcare Diagnostics Inc., Tarrytown, NY (US)

(21) Appl. No.: 13/635,914

(22) PCT Filed: Mar. 17, 2011
(86) PCT No.: PCT/US11/28768

§ 371 Date: Nov. 14, 2012

Related U.S. Application Data
(60) Provisional application No. 61/315,482, filed on Mar. 19, 2010.

(51) Int. Cl. A61B 19/00

(52) U.S. Cl. CPC A61B 19/56
USPC 606/1

(57) ABSTRACT

A workstation architecture for one or more medical diagnostic instruments is provided using a modular approach. Elements common to operations for the instruments are grouped together as a set of service components that are made available to instrument specific software applications. Application developers can develop software specific to the instrument while accessing the common service components to speed software development. A user interface tool is provided to permit a customized user interface to be developed for the instrument, within a generally consistent interface environment. The resulting user interfaces have a consistent look and behavior among different instrument types to facilitate a simplified and familiar user experience. The common service components provide a broad variety of services that are useful to developers and are easily integrated with instrument specific software. Features such as language variability and secure access points are provided in a distributed environment.

### STATEMENT REGARDING FEDERALLY SPONSORED RESEARCH OR DEVELOPMENT

N/A

### BACKGROUND OF THE INVENTION

The present disclosure relates generally to workstation implementations for medical diagnostic instruments, and relates more particularly to a modular workstation architecture for interoperation with one or more medical diagnostics instrument.

Medical diagnostic instruments are typically complex with a significant amount of detail involved in specifying and conducting diagnostic tests. In addition, numerous types of diagnostic instruments exist, each type typically including detailed operating specifications that are distinct among the different types. As a result, workstation software applications constructed to manage different types of instruments tend to be unique and individualized for the instrument. Development of such workstation software applications is often conducted independently for each type of instrument for at least this reason.

Providing an independently customized software application for a workstation connected to a particular instrument has several notable drawbacks. For example, it may be desirable to customize workstation software applications for different sites, geographic locations or industries, which is difficult if not impractical to achieve with application software that is specific to a type of instrument. For example, different versions of the instrument specific application software would be created and maintained, leading to cumbersome costs, complex maintenance tracking and lack of revision consistency among different installations. These drawbacks are amplified by the number of different instrument types and workstation platforms for managing the different types of instruments.

Moreover, when an instrument is updated with improved techniques, components or software, the accompanying workstation software is typically updated as well. Such an update to the workstation software application is specific to the instrument, so that developers of the software application or those familiar with the software application make software revisions that are unique to the given software application. Otherwise, a developer who is unfamiliar with the software application typically has a significant learning curve to understand the unique aspects of the software application to be able to perform the desired revisions and updates, which leads to extended development or revision cycle time.

Because of the complexity and detailed specifications involved in medical diagnostic instruments, the software development approach for developing a workstation software application is typically conducted on a one-on-one basis, so that a unique workstation application is developed for each unique instrument type. This convention typically means that workstation applications are developed for new instrument platforms on an ongoing basis. In addition, different functionalities were often developed by different development teams, resulting in different interfaces, operation, use of different programming languages, and often different workstation application platforms. These efforts were typically repeated and duplicated in parallel among the different instrument types, meaning that more development time, testing time and resources would be used in the development process cycle.

Furthermore, the user interface of the workstation would typically have a different presentation to the user among the different instruments, leading to an inconsistent user experience among the different instrument workstation platforms. This type of inconsistency among workstation applications leads to workstation experiences that are unique with regard to training, support and maintenance, so that there is little or no opportunity to take advantage of commonalties among the instrument workstation platforms.

Moreover, implementing updates or improvements on a consistent basis is highly complex and consumes significant resources. As an example, an instrument manufacturer may desire to have a common feature provided in a workstation application among the different types of instruments. In such an instance, each type of instrument workstation application is separately updated in accordance with the unique architecture and user interface of the workstation application for the given type of instrument. Accordingly, even a relatively simple common update among different types of instruments can represent a significant challenge in developer time and implementation costs.

### BRIEF SUMMARY OF THE INVENTION

In accordance with the present disclosure, a modular instrument workstation architecture for software applications is provided to overcome at least some of the drawbacks of the conventional systems. The architecture provides a data manager component that operates as a common central manager for operations that are common to workstation functions. The data manager exposes various interfaces to permit calls to be made by other system components that may be instrument specific. For example, an instrument driver specific to an instrument implementation can interact with the data manager to obtain a consistent common structure for workstation functionality among different types of instrument implementations.

A user interface interacts with the data manager and the instrument driver and permits user input and output at the workstation for controlling and managing different aspects of the workstation or instrument, or for obtaining information exchange among the various system components. The user interface has some generalized components that are common among workstation implementations, as well as instrument specific components that are developed for a specific instrument. The user interface presents a display that is configurable for the specific instrument, while providing a consistent user experience among different types of diagnostic instruments.

As part of the modular workstation architecture, a workstation framework for use with development of instrument specific software is provided. The workstation framework for the development of instrument specific software provides a rich set of reusable software components and libraries that instrument workstation developers can access and use to develop software that is specific to a given instrument for managing instrument operations. The workstation framework components can be used by software developers for specific instrument application development to include business logic and user interface products that are easily integrated with the overall workstation architecture. With the workstation framework, developers for instrument specific software are provided with a means to develop software that is customized to an instrument, within the structure of the overall workstation architecture. The workstation framework thus permits individualized instrument software development, within a workstation architecture that is consistent across different instrument workstation platforms.

According to another feature of the modular workstation architecture, a user interface shell is provided, which acts as a configurable user interface host. The user interface shell provides a user interface display that is configured with XML to obtain a desired look and feel, such as in the configuration of window layouts, component sizes and fonts, for example.

According to a feature of the user interface shell, the configuration of the display can be modified by reconfiguring the XML statements used to describe the user interface display. The XML code can be modified to provide a different display configuration, where the XML code is interpreted to obtain a modified display without having to recompile source code. The use of XML code also permits user interface functionality to be extended or modified with the addition of command objects to an XML configuration file. With this approach, the user interface can be customized for different sites or customers, on location, without compiling or recompiling source code. The user interface shell thus provides a configurable host instrument management user interface that is integrated into the modular workstation architecture to permit a consistent look and feel across different instrument platforms.

A number of tools and features to assist the development, configuration and integration processes are provided in the modular workstation architecture. A model data manager user interface is offered to provide a prebuilt user interface for data manager features that can be integrated in an instrument workstation to permit a consistent user interface look and feel across different instrument platforms. A configuration management tool is provided to facilitate configuration setup and modification for service components in the workstation architecture.

According to another feature of the modular workstation architecture, a user interface shell designer tool is provided for simplifying the design and configuration of the user interface shell. A developer can use the user interface shell designer tool to configure XML code in accordance with desired configuration parameters. For example, a field service engineer can configure or reconfigure the XML code used with the user interface shell in accordance with customer specifications on location, without having to write or rewrite a detailed XML code. In addition, the user interface shell designer tool permits reconfiguration of the user interface shell without compiling or recompiling source code. The user interface shell designer tool also provides features that include a preview function to permit changes to the user interface to be screened prior to being committed.

According to another feature of the modular workstation architecture, security controls are provided to implement role based user interface security access points. A user interface control is marked as being a candidate for security by including a security attribute during the creation or formation of the UI control. The user interface shell provides a link between the securable user interface control and the feature or property that has secure attributes. For example, a user interface control may have securable properties such as enable, visible or mask among the different security configurations for the control. When the user interface shell loads the control, the security properties of the control features are implemented based on the user permission and the security configuration of the control feature. The control feature properties can be assigned in an XML configuration file, so that modifications to the security features for the controls can be made without compiling or recompiling source code. With this approach, a user is provided with predetermined security access points in the user interface in accordance with a given user permission level.

According to another feature of the modular workstation architecture, localization support is provided to enable different languages or features that are specific to a given locale. The user interface shell provides a local customization feature that can be selected to modify the language used with the user interface in real time, that is, without restarting the user interface application. With this feature, multiple language support is provided that enables users to rapidly switch language and other local data to increase the usability of the user interface.

### BRIEF DESCRIPTION OF THE SEVERAL VIEWS OF THE DRAWINGS

The present disclosure is described in greater detail below, with reference to the accompanying drawings, in which:

FIG. 1 is a block diagram of component connectivity in a network utilizing the modular workstation architecture;

FIG. 2 is a diagram of a data manager server;

FIG. 3 is a schematic block diagram of a data manager service illustrating service components;

FIG. 4 is a process flow diagram illustrating message handling in the data manager of the present disclosure;

FIG. 5 is a process flow diagram illustrating establishment of communication connectivity between a data manager service and an instrument manager service;

FIG. 6 is a process flow diagram illustrating a security authentication process; and

FIG. 7 is a process flow diagram illustrating security authentication from a webpage.

### DETAILED DESCRIPTION OF THE INVENTION

This application claims the benefit of U.S. provisional application No. 61/315,482, filed Mar. 19, 2010, the entire disclosure of which is hereby incorporated herein by reference.

The present disclosure provides a universal instrument workstation that has a modular architecture and features multi-user support, remote accessibility, core common components and instrument specific components. The modular architecture provides a development platform for instrument management software that is specific to a given instrument, which is integrated into an overall workstation architecture that features common components that are usable with different instrument platforms. The modular architecture arrangement permits rapid development of instrument specific applications within an architecture that is known across multiple different instrument platforms to obtain an instrument workstation user interface that provides a similar and familiar user experience with different instrument platforms. The modular architecture permits rapid development of instrument specific workstation software, while being more easily maintainable and less complex in implementation.

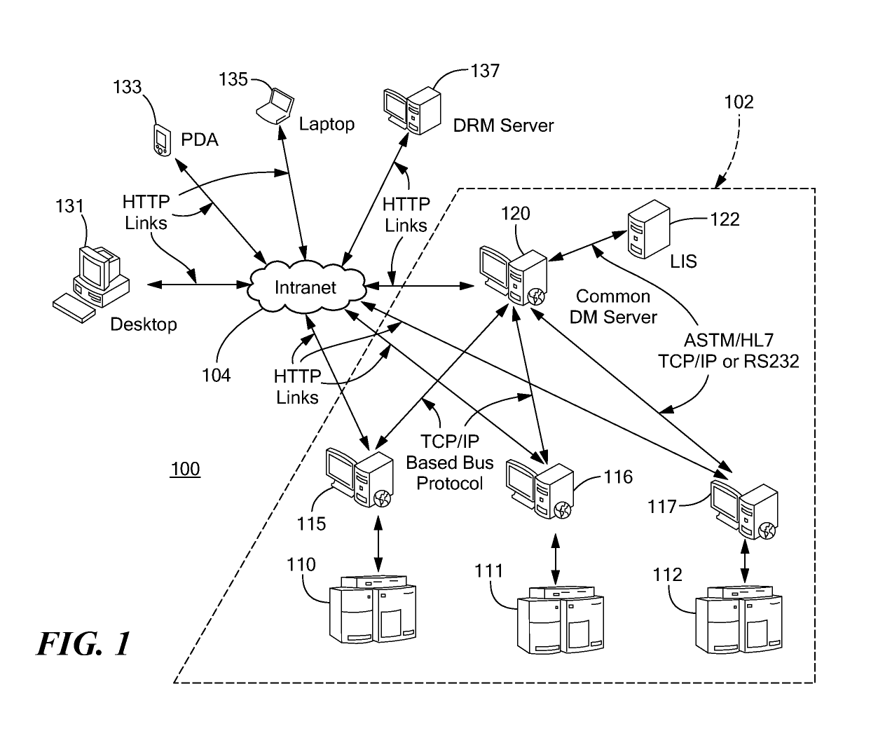

Referring now to FIG. 1, a network 100 of devices associated with medical diagnostic equipment is illustrated. Network 100 includes an intranet 104 that operates through various devices within a laboratory 102 and devices that are external to laboratory 102. External devices, such as a desktop computer 131, a personal digital assistant (PDA) 133, a laptop computer 135 or a digital rights management (DRM) server 137 may communicate with each other or with devices in laboratory 102 through intranet 104. Typically, communication within intranet 104 may take place over links that provide a transport protocol for communicating HTTP formatted information. These HTTP links can thus provide formatted data for sharing between various devices connected through intranet 104.

Within laboratory 102, various medical diagnostic instruments are setup for operation in a workstation configuration. For example, diagnostic instrument 110, 111 and 112 may be different types or the same type of medical diagnostic instrument, which may be managed within a workstation configuration through computer workstations 115, 116 and 117, respectively. Computer workstations 115-117 permit a user to interact with respective instruments 110-112 and manage data and control information associated with instruments 110-112. In accordance with the present disclosure, workstations 115 and 116 provide instrument management (IM) functionality, which may include instrument drivers for instrument control and data exchange for operating respective instruments 110, 111. Workstation 117 is illustrated as a legacy workstation that can be integrated into the overall modular architecture of the present disclosure even though workstation 117 may not implement modular components of the disclosed modular architecture. That is, workstation 117 may be implemented as a uniquely programmed stand alone device for use with instrument 112, and may not be able to take advantage of the commonalties provided by the presently disclosed modular architecture. Nevertheless, workstation 117 can be integrated into the network and system shown in network 100 of FIG. 1, to take advantage of the core functionality provided by a common data manager (DM) server 120.

Workstations 115-117 can communicate with DM server 120 over network links that may implement different types of protocols. For example, the links between workstations 115, 116 and DM server 120 can be implemented as a TCP/IP based bus protocol, while the link between workstation 117 and DM server 120 can carry messages based on the ASTM or HL7 standards using a TCP/IP transport protocol or an RS232 serial link. This type of communication link is somewhat specific in nature to the exchange of information in the medical services community, so that legacy systems, such as workstation 117 may be tied into using such a communication link. This type of communication link may also be used between DM server 120 and a laboratory information system (LIS) 122 that supports various types of laboratory processes in the medical services industry. The connectivity and configuration of laboratory 102 is provided as an example, and may be varied depending upon instrument configuration and accessibility to data as desired. However, the main components of the presently disclosed modular architecture are implemented through DM server 120 and workstation 115 or 116 in conjunction with the respective instruments 110 or 111.

DM server 120 implements the modular components associated with a data manager in the modular architecture of the present disclosure. The data manager provides an array of data management features that an instrument can use with instrument workstation software, such as may be implemented on workstations 115 or 116. The data manager components implemented in DM server 120 seamlessly integrate with instrument manager components implemented on workstations 115, 116 to provide a complete feature set for instrument workstation management. The instrument management software implemented on workstation 115, 116 is specific to the associated instruments 110, 111, while also providing access to common core features implemented with DM server 120. According to some exemplary embodiments, an instrument workstation, such as workstations 115, 116, may be implemented to include a data manager service as a stand alone unit. That is, an instrument workstation can be implemented to include the entirety of the modular architecture components of the present disclosure to operate as a single unit, rather than distributing modular architecture functionality among different devices, as is illustrated with workstations 115, 116 and DM server 120 in FIG. 1.

### Data Manager

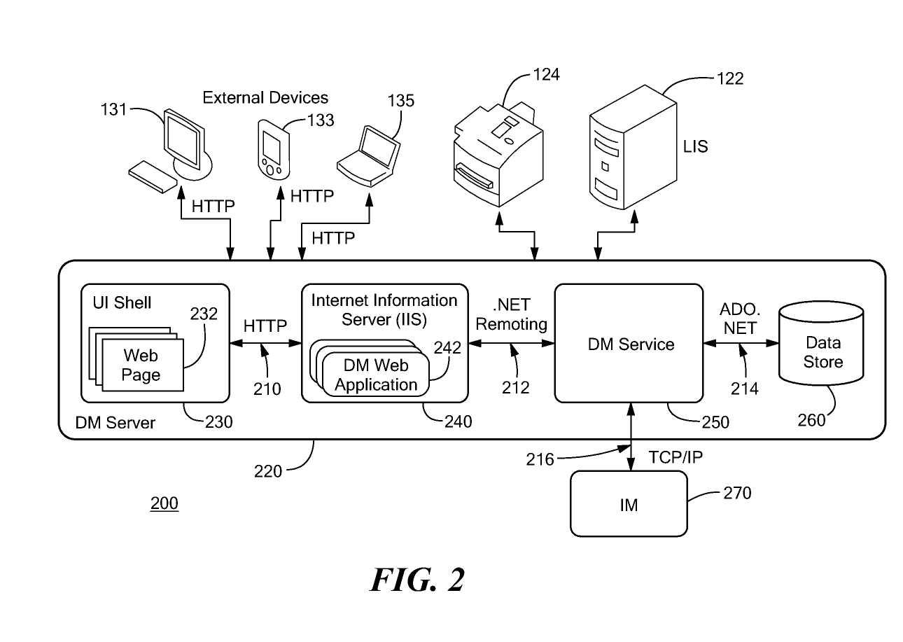

Referring now to FIG. 2, a diagram of a modular architecture 200 in accordance with the present disclosure is illustrated. Architecture 200 includes a DM server 220 and an instrument manager (IM) 270. DM server 220 may be implemented as DM server 120 illustrated in FIG. 1, while IM 270 may be implemented on workstation 115, 116 as an instrument manager, as discussed above.

DM server 220 includes a number of general components connected through various interfaces for providing core services in accordance with the presently disclosed modular architecture. The components of DM server 220 illustrated in architecture 200 include a user interface (UI) shell 230, an internet information server (IIS) 240, a DM service 250 and a data store 260. UI shell 230 is coupled to DM service 250 through IIS 240, over an HTTP link 210 and a .NET remoting connection 212. UI shell 230 provides a user interface for interaction with external devices, such as desktop 131, PDA 133 or laptop 135. Users can access UI shell 230, and receive web pages 232 over a link, illustrated in architecture 200 as an HTTP link. Accordingly, UI shell 230 permits local and remote access to instrument information, and is provided within the constraints of the presently disclosed overall modular architecture.

Web pages 232 can be provided and modified through IIS 240 using a data manager (DM) web application 242. DM service 250 provides an interface to the instrument (not shown) through IM 270, so that the instrument can be managed through UI shell 230. The user receives information in a display and can provide instructions to the instrument through UI shell 230 and web pages 232. Commands and data from UI shell 230 are exchanged through HTTP link 210 and IIS 240 through DM web application 242. The instructions and data are further exchanged with DM service 250 over .NET remoting link 212 for eventual data exchange and management of the instrument through IM 270.

The arrangement of UI shell 230 to be accessed through IIS 240 is a modular approach to permit UI shell 230 to provide remote access, or a local user interface at a workstation in the locale of the instrument being managed, for example. That is, DM server 220 may be implemented on a workstation that is local to the instrument being managed with the modular architectural approach. In addition, DM server 220 provides connectivity and services for a printer 124 and an LIS system 122 to permit a complete infrastructure for managing medical diagnostic instruments and the data associated with the instrument, procedures or patients.

DM server 220 hosts DM service 250, which provides common core services for the disclosed modular workstation architecture. DM service 250 has access to a data store 260, which may be, for example, a relational database accessed through ADO.NET link 214. DM service 250 also provides information and instruction exchange to UI shell 230, as discussed above, and hosts services that are usable by IM 270 over a TCP/IP link 216. DM service 250 provides a number of services that are common to the workstation architecture, so that developers of instrument software need not recreate application services with each new instrument platform or instrument or software version. Some of the services provided by DM service 250 include: instrument communication services; external device communication services; user services; business serves; data store services; common user interface shell; and a dashboard service that permits access to multiple instrument managers and legacy platforms.

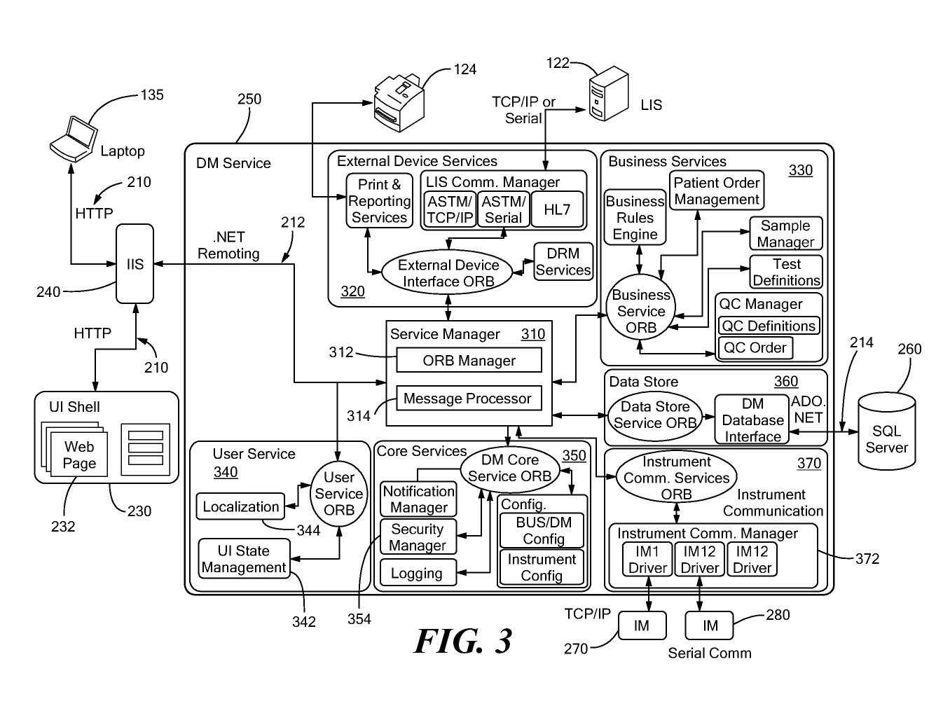

Referring now to FIG. 3, a diagram of DM service 250 is illustrated in greater detail. DM service 250 is illustrated as having a number of logical service blocks including external device services block 320, business services block 330, user service block 340, core services block 350, data store block 360 and instrument communication block 370. DM service 250 also includes a DM service manager 310 that coordinates the services provided by each of the logical service blocks.

Service manager 310 includes an object request broker (ORB) manager 312 and a message processor 314 for carrying out the management and control functions of service manager 310. Aside from being in communication with the different logical service blocks contained within DM service 250, service manager 310 includes a .NET remoting link 212 for communication with IIS 240 to provide web application support for the one or more workstations associated with DM service 250. ORB manager 312 provides management services for the various subsystems of DM service 250, as well as object discovery across application domains.

ORB manager 312 serves a number of functions for contributing to managing the various operations provided in DM service 250. The object discovery service across application domains permits remote applications to locate configured services through service manager 310. For example, a remote process or machine can access the logical service blocks of DM service 250 by invoking ORB manager 312. ORB manager 312 includes a configuration manager that is used to load subsystem services based on a service configuration. When subsystem services are loaded, for example, business services 330 are loaded for a user, ORB manager 312 implements the loading of the subsystem service and manages the service through its lifetime, that is, while the subsystem service is loaded. ORB manager 312 also permits discovery of services by remote processes or remote machines, for example, to permit users to access features in DM service 250 that may be updated or modified. The configuration manager provided with ORB manager 312 is used to set the configuration of services to permit various options for loading services in accordance with the configuration. Service components may therefore be loaded based on a configurable setting using the configuration manager. This arrangement permits instrument managers, such as IM 270 or 280, to take advantage of updated configurations and discover and utilize updated services or components within DM service 250.

Message processor 314 in service manager 310 handles messages routed to components in DM service 250. Messages have a particular structure for routing among the different components of DM service 250, and may include a source and destination as well as a priority and other identifiers or attributes of the message. Message processor 314 may implement priority message queuing and handle synchronous and/or asynchronous message dispatching. Some messages received by message processor 314 may be processed by locating a handler for the message.

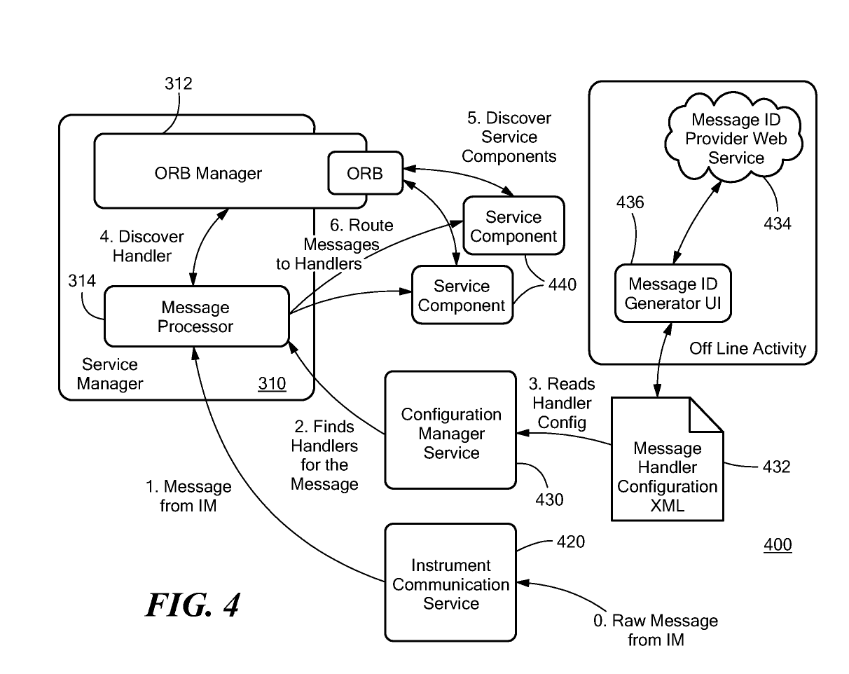

Referring now to FIG. 4, a process flow 400 illustrates the processing of a message received by message processor 314 that uses a handler for the message. In process flow 400, a message is generated from an instrument (now shown) and communicated to service manager 310 by an instrument communication service 420 operating within an instrument manager, such as IM 270 (FIG. 3). The message from the instrument manager is delivered to message processor 314, which in turn seeks a handler for the message using configuration manager service 430.

Configuration manager service 430 provides information on the configuration of various components of the modular architecture, including message handlers for processing messages. Configuration manager service 430 is implemented to read configurations for handlers, based on an XML code 432 describing the message handler configuration. XML code 432 is setup and configured offline in accordance with the desired processing for the given message. For example, a message ID can be generated from a message ID provider web service 434 and provided to a message ID generator user interface 436 for implementation in XML code 432. The generation of a message ID from web service 434 and user interface 436 may be done offline, and may be orchestrated or controlled by configuration manager service 430. For example, configuration manager service 430 may be used to generate XML code that includes message handler configuration information. The facility of using XML code 432 to determine the message handler configuration for the incoming message permits an offline configuration that does not require compiling or recompiling source code to modify the system settings in response to an incoming message.

Once the message handler configuration is determined by configuration manager service 430, the handler is located by ORB manager 312, which has the facility to discover service components in the application domain of DM service 250 (FIG. 3). Once the appropriate service components 440 are discovered, message processor 314 routes the messages to the discovered service components 440 to permit handling processing for the message.

The use of ORB manager 312 to discover service components, and the use of configuration manager service 430 coupled with XML code 432 provides a flexibility for DM service 250 that allows extensibility and modification of DM service 250, without requiring modifications to an instrument manager that communicates with DM service 250. Using this flexible arrangement, modifications and updates to DM service 250 can be carried out across numerous different platforms to update or provide additional services or components, without custom modification for each instrument platform. Since each logical service block of DM service 250 (FIG. 3) includes an ORB module, service components can be added, modified or removed in DM service 250, and located using ORB manager 312 in service manager 310. To an instrument manager, such as IM 270, DM service 250 can thus appear as a framework of APIs that provide various services to the instrument manager.

Configuration manager service 430 resides within DM service 250 and provides a mechanism for managing configuration of various components in the disclosed modular architecture. For example, configuration manager service 430 can be used to manage configurations for components of DM service 250, IM 270 or UI shell 230. The configurations tracked by configuration manager service 430 are maintained with individual configuration files per each component setting. Configuration manager service 430 draws on XML files to establish configuration settings for the various components for which configuration management is provided. A user interface tool is provided to access configuration manager service 430 to enable configuration of various settings for components. For example, developers can work directly with object class attributes using the user interface tool for the configuration manager. The class attributes can be stored in XML format upon creation of object instances, and the XML format can be used by configuration manager service 430 to determine configuration settings for instances of logical service components. The XML code can be modified using configuration manager service 430, or can be modified using an XML editor. As described above, the use of configuration manager service 430 and the associated XML files provides a flexibility for features that can be invoked from the instrument manager without having to update the instrument manager or compile or recompile associated source code.

Referring again to FIG. 3, DM service 250 provides a logical service block for instrument communication 370, which hosts an instrument communication manager 372. Instrument communication manager 372 is a component that is common to the data manager and instrument manager to permit the different domains to have reusable components, as well as providing a common format for exchanging messages between the DM and IM. When the instrument communication manager is implemented in DM service 250, it provides communication management for all connected IMs, such as IM 270 and 280, illustrated in FIG. 3. In such a configuration, DM service 250 can be used with multiple instrument platforms. When the instrument communication manager is located in an instrument manager, such as IM 270 or 280, it manages communications with the connected data manager, such as DM service 250 illustrated in FIG. 3. In addition, the instrument communication manager implemented in an IM manages communications with the instrument as an analytical module (AM) to permit the exchange of messages with the instrument. The instrument communication manager supports a number of different protocols that might be used by a specific communication driver interface. For example, FIG. 3 illustrates instrument communication manager 372 being used to communicate over a TCP/IP link or a serial communication link with IMs 270, 280, respectively.

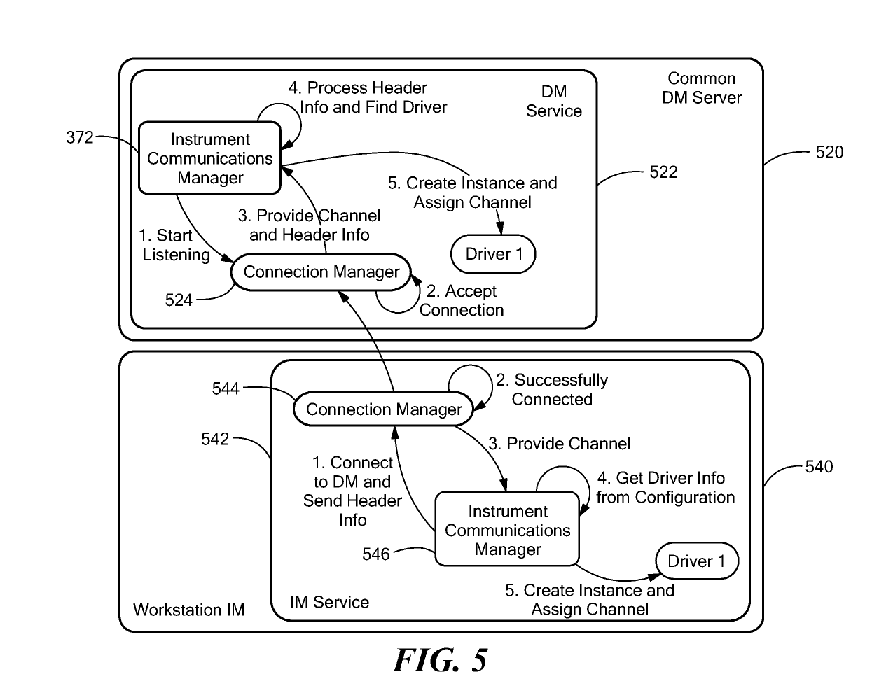

Like other logical service components in DM service 250, instrument communication manager 372 can be configured to have various communication drivers based on XML code that can be modified or updated offline. Referring to FIG. 5, a process flow for establishing communication between a DM server 520 and a workstation IM 540 is illustrated. DM server 520 includes DM service 522, which can be implemented as DM service 250 (FIG. 3). Workstation IM 540 similarly includes IM service 542 that parallels DM service 522 for communication management between a data manager and an instrument manager.

The process flow illustrated in FIG. 5 begins with instrument communication manager 372 instructing a connection manager 524 to listen for a connection. A corresponding connection manager 544 hosted by IM service 542 broadcasts messages for connection to connection manager 524. Once a connection is established between connection managers 524 and 544, instrument communication manager 546 provides header information to indicate a type of driver or protocol to be used in the communication setup. Connection manager 524 forwards the header information to instrument manager 372 in DM service 522, and also provides communication channel information to setup a communication channel. Similarly, connection manager 544 provides a channel to instrument communication manager 546 to permit DM service 522 and IM service 542 to communicate over a common channel. Once the channel is established, instrument communication manager 372 processes the header information received from instrument communication manager 546 and locates a communication driver for the chosen communication type or protocol. In parallel, instrument communication manager 546 obtains configuration information concerning the communication driver to be used for communicating with DM service 522. Instrument communication managers 372 and 546 each create instances of a communication driver for handling communication between respective DM service 522 and IM service 542. The object instance created in each of DM service 522 and IM service 542 are assigned an attribute for the channel provided by the respective connection managers 524, 544. Communication between DM server 520 and workstation instrument manager 540 can then take place through the respective driver object instances.

The modular arrangement of instrument communication manager 372 (and 546) permits DM service 250 (FIG. 3) to support a number of drivers simply by adding a driver to the configuration of DM service 250, which can be implemented using the configuration manager discussed above. Accordingly, developers for instrument management workstations can select a desired protocol for communication with the data manager service without placing restrictions on the data manager. In this way, instrument management platforms can use legacy communication protocols or systems, which can be supported in the data manager to avoid rewriting or recompiling source code to accommodate a particular communication driver set. Moreover, because the communication driver configuration in DM service 522 (or DM service 250) is based on configurations provided in XML coded files, modifications to communication driver configurations can be implemented without a need to reconfigure, rewrite, or recompile software, while permitting the use of additional or new communication drivers that can be selected by the instrument management platform in accordance with the process described in FIG. 5.

Referring again to FIG. 3, DM service 250 provides external device communication that can be implemented in the same way as described above for an instrument management platform. That is, various drivers can be provided in external device services 320 for printers, such as printer 124, LIS servers, such as LIS server 122, as well as DRM services. The variety of communication drivers that can be made available in external device services 320 permit communication with legacy type equipment including LIS server 122, for example.

The arrangement and organization of the disclosed modular architecture for managing medical diagnostic instruments provides a number of advantages including a rich variety of services that are accessible by an instrument specific workstation platform, reusable frameworks for implementing data management and instrument management workstations and component configuration that avoids revision of software or recompiling of source code. Examples of these features and advantages can be seen in the variety of services provided by DM service 250, which are available to multiple instrument management platforms simultaneously. In addition, the various logical service blocks and components of the presently disclosed workstation architecture can be provided to an application developer that seeks to integrate instrument management software with the overall workstation architecture. The application developer can access reusable software components and libraries available in DM service 250, for example, including business services 330, user service 340 and core services 350 to implement an instrument management platform.

Application developers can take advantage of the framework of common services, as well as a framework of software components available for development of the instrument management platform. For example, application developers can be provided with a user interface shell that can be used to host executable software (binaries) related to the instrument in a format that is standardized for medical diagnostic instruments of different types. That is, the application developers are provided with a user interface shell that adopts certain conventions for items that are generally common to different types of instruments.

The user interface shell may, for example, provide selectable components or tabs for items such as work folders, test results, quality control, setup, utilities or events, each of which may have child screens that are arranged by tabs that are categorized under the parent tab. The application developer can setup various features and displays related to the instrument workstation platform under development, while providing a common organization structure that is at least somewhat consistent across different instrument workstation platforms. Accordingly, the application developer has a reusable framework of components that foster rapid development of instrument workstation software that is unique to an instrument, while obtaining a look and feel that provides a consistent user experience.

Another advantage provided by the features of the presently disclosed modular architecture discussed above is the configurable nature of the various components and services provided in DM service 250. Configurable components can be accessed through a configuration manager service and manager user interface to perform a number of configuration functions in accordance with several features and advantages of the present disclosure. For example, as discussed above with respect to the configuration manager service 430, XML files can be created and modified to define the configuration for various service components. The XML files can be modified by configuration manager service 430 or with an XML editor, which can be undertaken offline by, for example, a field service engineer. Similarly, instrument communication manager 372 can rely on XML files to define drivers that can be implemented on a plug and play basis, so that modification or implementation of a given driver is a matter of forming or editing an XML file, rather than programming and compiling source code to create or modify a driver or service component configuration. The advantage of configuring workstation components using XML code is also applied in the formation of a user interface through the user interface shell 230.

### UI Shell

UI shell 230 is a configurable user interface host that displays components in accordance with user defined XML definitions. The user interface provided through the UI shell can contain window layouts, components, locations and sizes of components and fonts that are configured using XML code. One advantage of using XML code to define the contents of the user interface is the potential for online reconfiguration of the user interface, without rewriting software or recompiling code to make changes to the user interface. Indeed, it is possible to modify the XML code that defines the user interface components and change the user interface on the fly, without reloading the user interface. The user interface functionality can be extended or modified by adding objects to the XML configuration file. These features and advantages of the configuration of the user interface shell permits locally customized user interfaces that can be modified in the field by a field service engineer, for example. The approach using XML code also is flexible for multiple projects, instruments and versions, as well as customers. In addition, the user interface shell can be constructed with components that obtain information from IM 270, for example so that the user interface shell can contain both DM and IM components.

Another advantage of UI shell 230 is the ability to host web pages 232 to provide an integrated windows and web user interface. In addition, UI shell 230 provides support for external user interface operations, as well as external binary execution. UI shell 230 provides foundation elements for organizing and managing window layouts to permit rapid prototyping of user interface displays and functionality.

UI shell 230 hosts a number of component conventions, including window types that can be multiple document interfaces (MDI) child windows, floating or pinned windows and mobile windows. UI shell 230 also supports frames that can be of the type of grid, flow, pixel, tab and/or collapsible. Standard window controls are also provided, including a shell title bar, shell browser, shell buttons, shell pages, web shell grid, shell label, and other types of standard window controls to provide a familiar user interface setting. User controls in UI shell 230 are designed in accordance with XML code, and are loaded and displayed from configured assemblies. The behavior of the controls is also loaded and set from configured assemblies through commands, which can also be configured in XML code. The definition for all the components of a display in UI shell 230 can be configured in an XML file to further organize and enhance the modular aspects of UI shell 230.

The configuration of UI shell 230 itself can be modified or determined using an XML shell configuration file. For example, configuration manager service 430 (FIG. 4) can load the shell configuration from an XML shell configuration file when a user invokes the user interface. Once UI shell 230 is configured, it can load user controls and execute loading events to implement controls and commands. Controls and commands can be defined to locate other controls and commands using a name feature, which can also be loaded using UI shell 230. Various types of global controls or attributes can be set with the shell configuration XML file, including colors, fonts, images, sizes and other shell configuration settings.

In accordance with a feature of the presently disclosed modular architecture, a shell designer tool is provided to facilitate creation and configuration of shell elements. The shell designer tool provides a tree structure to simplify viewing of shell elements in a hierarchy, and can provide previews of user interface displays as configuration entries are added or modified. The shell designer tool permits simplified creation and attribute setting for user interface components, including commands for executing operations within the user interface. The shell designer tool is also useful for displaying and configuring web user interface elements that can be provided to remote entities over HTTP links, for example. The shell designer tool establishes XML code to describe the configuration of the various user interface components and commands. The XML code can be modified by the shell designer tool in an easy to use format to reduce the complexities of XML coding. However, the XML files that describe the user interface components and commands can also be edited directly, as may be suitable for conducting bulk edits. In addition, the configuration user interface that handles configuration for service components, as described above, can be used to modify UI shell components and commands, which can result in a more expedient configuration modification.

UI shell 230 can be used at a local workstation, to implement a user interface. UI shell 230 thus provides a flexible and convenient mechanism for accessing DM service information and IM information at a local workstation, remote workstation or through external devices that can be coupled to DM server 220 (FIG. 2) through an internet link, which may be implemented using HTTP.

Referring again to FIG. 3, user service 340 includes UI state management 342, which helps to manage web application sessions and event changes within DM server 220. UI state manager 342 can operate with multiple simultaneous web clients and provides updates that can be based on polled state changes. For example, if there is a notification of an event state change, as may be noted in business services 330, for example, a state change notification is generated to UI state manager 342. UI state manager 342 modifies a stored state related to a particular web session hosted in UI shell 230. The web session polls the state maintained by UI state manager 342 to determine if an update is needed, and refreshes the web user interface accordingly.

User service 340 also includes localization manager 344, which provides localization services for DM service 250. Localization manager 344 supports a number of different languages for the user interface, as well as other local configuration information such as time and date as well as other local conventions. Localization manager 344 permits selection of different localization characteristics in real time without requiring a restart of the user interface or other components of the modular architecture. The notification of other components of DM service 250 is also accomplished through localization manger 344, so that locale specific resources can be loaded and provided to user interfaces, for example.

Another feature of the presently disclosed modular architecture related to user interface implementations is a dashboard service that permits remote access to data available from an instrument manager. The dashboard service enables read-only access to certain instrument manager related data from any other connected instrument manager, data manager and other devices connected to a local intranet. The data manager provides the infrastructure to access data from all instrument managers in a single location. Each instrument manager can provide a selection of data based on the configuration of the individual instrument managers. The dashboard service permits legacy machines to easily join in data sharing by providing access to the data on an intranet or web enabled service.

### Workstation Security

Core services 350 illustrated as part of DM service 250 includes a security manager 354. Security manager 354 provides a number of security related services for the presently disclosed modular architecture to prevent unauthorized access to objects and data within the workstation and network environment. Some of the services provided by security manager 354 include authentication service, authorization service, user management, role and/or group management, policy management and implementation of regulatory requirements. As part of the authentication service, security manager 354 authenticates messages from instrument managers, such as IM 270 received at DM service 250. In addition, authentication for .NET remoting messages is also provided, where a call or messages are received from UI shell 230 or a web application.

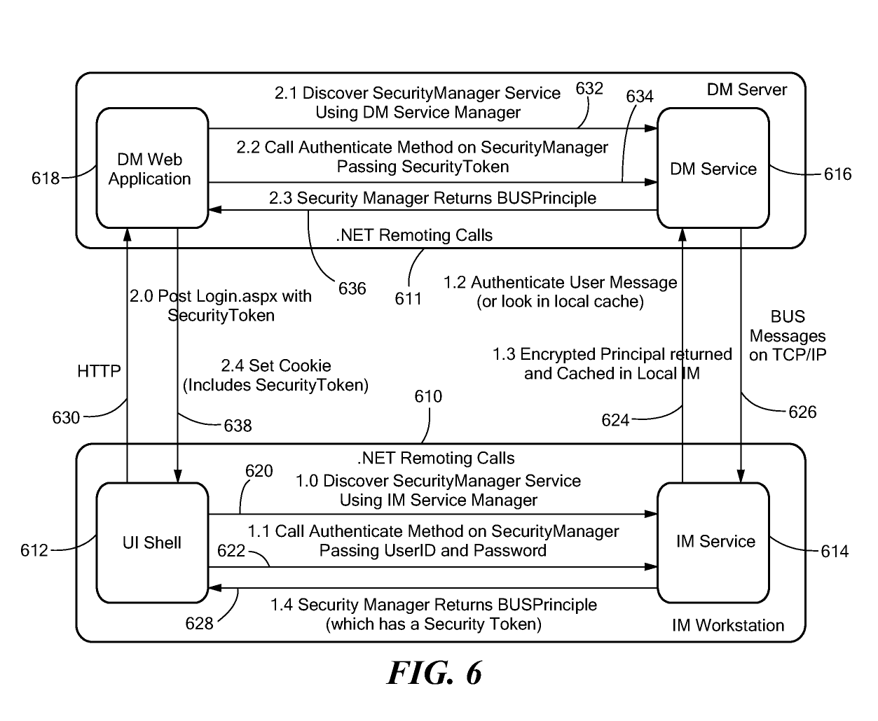

Referring now to FIG. 6, a diagram of a security process according to an exemplary embodiment of the present disclosure is illustrated. The illustrated process for security management begins by utilizing a feature of the presently disclosed modular architecture in step 620 by discovering security manager services using the instrument manager service manager. The IM service manager (not shown) is similar in structure and operation to security manager 354 provided in core services 350 of DM service 250 (FIG. 3). The security manager service is discovered using the ORB manager (not shown) implemented in IM workstation 610. The ORB manager implemented in IM workstation 610 operates similarly to ORB manager 312 (FIG. 3) discussed above for discovering and loading services in conjunction with a configuration manager service that draws on configuration information provided in XML code. IM workstation 610 illustrates messages passed between a UI shell 612 and IM service 614 using .NET remoting calls.

Once the security manager services are discovered in step 620, UI shell 612 calls an authenticate method on the security manager object and passes a user ID and password to IM service 614. The user ID and password provided by UI shell 612 are entered by the user upon logging into the system to permit secure access to IM workstation 610. Once IM service 614 receives the user ID and password in step 622, a message is sent to DM service 616 residing in DM server 611. Message 624 is sent over a TCP/IP link connecting IM service 614 and DM service 616 to authenticate the user message containing the user ID and password. DM service 616 responds to IM service 614 with message 626 then includes an encrypted component with a security token. IM service 614 passes message 628 to UI shell 612 with the encrypted component that includes the security token. UI shell 612 then can submit a message 630 to DM web application 618 over an HTTP link, which message includes a security access login with the security token obtained from DM service 616. DM web application 618 can then provide message 632 to DM service 616 to discover security manager services in DM service 616 using DM service manager 310 (FIG. 3). The security manager service 354 is discovered and loaded in accordance with the configuration of the security manager service, which can be determined using XML code definitions as described above.

Once the security manager service in DM service 616 is discovered, DM web application 618 can deliver message 634 to security manager service 354 in DM service 616 to implement a call on the authenticate method available in security manager service 354. The call of the authenticate method includes the passing of a security token that was obtained from DM service 616, through IM service 614 and UI shell 612 to verify the user and authenticate security access. DM service 616, through security manager service 354, returns message 636 to DM web application 618 with a security component that verifies the authenticity of the user ID and password. DM web application 618 provides a message 638 to UI shell 612 that sets a cookie that includes a security token for UI shell 612. The user accessing IM workstation 610 through UI shell 612 is then authenticated through DM service 616 and is given access to DM service 616 and IM service 614 in accordance with the permissions set for the user.

The permissions and access levels set for the user are implemented in DM service 616. By setting permissions and access levels for the user, certain features of IM workstation 610 and DM server 611 may be available or unavailable, visible or non visible, or have other features enabled or disabled in accordance with the security level and permission of the user.

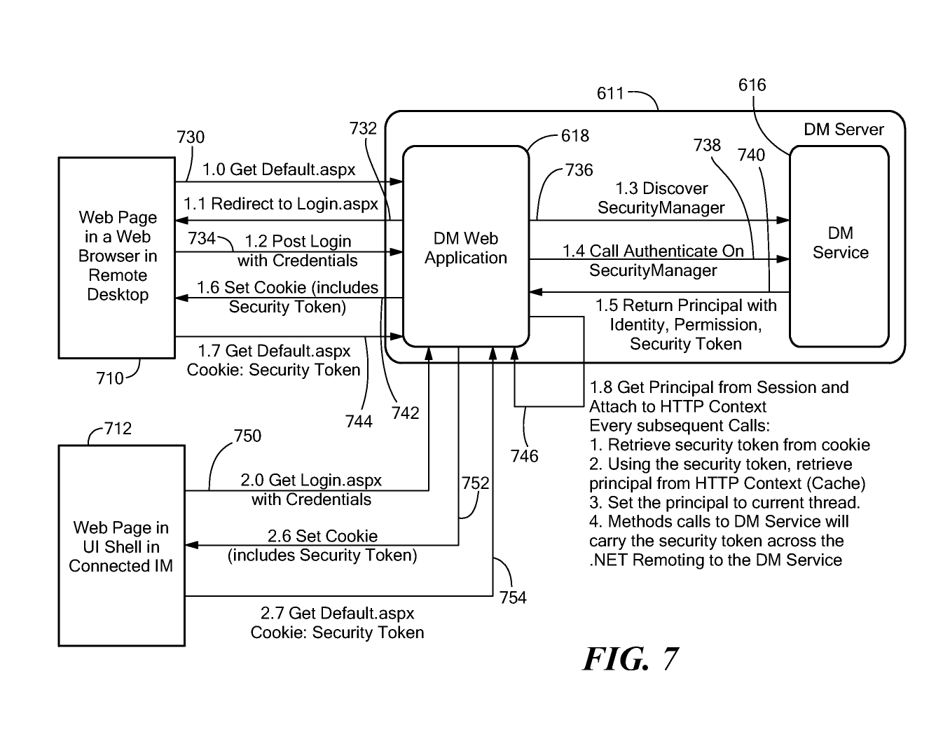

Referring now to FIG. 7, authentication of a user seeking access through a web application is illustrated. The remote user seeks to load a webpage from a remote desktop 710, for example, which prompts message 730 to be sent to DM web application 618 in DM server 611. Message 730 causes DM web application 618 to provide a default login page to desktop 710 in message 732. The user enters a login ID and password, or other security credentials, which are sent to DM web application 618 in message 734. DM web application 618 provides message 736 to DM service 616 to discover security manager services in DM service 616. The security manager services are discovered and loaded in accordance with a configuration provided in DM service 616, as described above.

Once the security manager service is discovered, DM web application 618 sends message 738 to DM service 616 to call an authenticate method on the security manager service. The security manager service processes the authentication method call using the logon ID and password or other security credentials provided to DM web application 618, and returns a message 740 to DM web application 618 that includes a security component including an established identity, permissions and a security token. DM web application 618 provides message 742 to desktop 710 so as to set a cookie that includes the security token. Desktop 710 can then submit message 744 to DM web application 618 to load web pages, frames, controls and/or commands in the current webpage of desktop 710. DM web application 618 receives the request from desktop 710 and attaches the security component to the HTTP context established for the session with desktop 710, as indicated in step 746.

As the user manipulates and moves about in the web application on remote desktop 710, calls made to DM web application 618 each include the cookie with the security token. Each call to DM web application 618 causes the security token to be retrieved from the cookie to permit the security component to be retrieved from the HTTP context. The call from desktop 710 is then given an execution thread to permit method calls to DM service 616 using the security token. The security token used for access to DM service 616 provides the user with access to data based on a permission or security level established in DM service 616.

The operations for secure access by a user accessing a web page in UI shell 712 are substantially the same as those described above for a user seeking secure access through a webpage in remote desktop 710. UI shell 712 provides message 750 to DM web application 618, to obtain a login with a user ID and password or other security credentials. In response, DM web application 618 discovers security manager services and calls an authenticate method on the security manager services located in DM service 616 as illustrated with messages 736 and 738. A security token is returned to DM web application 618 from DM service 616, which in turn prompts message 752 to UI shell 712 to provide a cookie that includes a security token. UI shell 712 can then provide the cookie with the security token to DM web application 618 in message 754 to access data in DM service 616, as shown in step 746.

The security features of the presently disclosed modular architecture permit conditioned access to system components based on the permissions associated with the user in accordance with group membership or security level settings for the user. Accordingly, the user permissions can be set in accordance with the user's logon ID or other security credentials using the security manager service. The security manager service provides security functions that include authentication, authorization, user management, role or group management, management of security policies and implementation of regulatory requirements for secure access to medical data, for example.

The process for creating modular security access points begins with the developer indicating whether a control is a securable entity by including a user interface security attribute with the control. This action marks the user interface control in the source code as a securable entity. The source code is compiled to provide a prebuilt operating image for the user interface so that the control with the security attribute is available for user interface design. The application developer can then create a control, such as “view patient demographics” based on the securable item that includes the security attribute. The instance of the user interface control can then be marked for behavior in accordance with security restrictions. For example, the “view patient demographics” user interface control can be specified for behavior for enable, visible and masked security restrictions. The application developer can also use the configuration service manager to specify XML code for the securable aspects of the control to provide behavior specification for full access, read only and no access privileges, for example. When the UI shell loads the control, the appropriate properties are applied, so that user permissions determine the security behavior for the control.

In addition, groups or user roles are established that determine the access permissions for a given user. User groups can be setup with particular access permissions, so that any member of the group receives those permissions upon logging in to the workstation. For example, a user group may be given full access permission to the control “view patient demographics” during a setup phase for user security and group membership, whereupon the users in that group receive full access to the information related to the control. Other user groups may have settings that provide read only access to the information associated with the control in accordance with the user group permission settings, for example.

Together with the user authentication processes described above, the secure user interface access points provided through a securable entity obtain a flexible and easily managed security mechanism for access to data and components of the workstation environment. The security properties for controls and commands can be configured using XML, so that software revision or source code recompilation need not be undertaken to manipulate the security property aspects of the controls and commands of the user interface. With this security construct, application developers can set up security on a customized basis for a given instrument implementation. The security facility operates under the modular approach, however, so that application developers need not be concerned with functions that are provided on a common basis for all instrument platforms. For example, the setting of security permissions for users and user groups is implemented using data manager services to obtain common settings for all connected instrument manager platforms.

The presently disclosed medical diagnostic instrument workstation architecture provides a number of advantages over prior configurations. Common functionality among different instruments is separated into a single logical and physical application. The single application can be developed once, tested once, and used repeatedly for new and different instrument platforms. The user interface provided with the application achieves a consistent user experience across multiple instrument platforms while providing an intuitive and easy-to-use presentation to assist users in achieving desired actions. The reusable framework library and source code provided to developers assist in speeding development of instrument specific software including instrument drivers. The architecture permits access to multiple connected computers within a laboratory or intranet. Revisions and maintenance of the common software elements is simplified and streamlined at a single resource point, rather than being distributed with respect to different instrument platforms and potentially different installations. All these features help to reduce development, testing and training efforts, while broadening the available services for the instrument workstation platform.

The operations herein described are purely exemplary and imply no particular order. Further, the operations can be used in any sequence when appropriate and can be partially used. With the above embodiments in mind, it should be understood that the disclosed systems, devices, methods and/or uses can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described herein that form part of the present disclosure are useful machine operations. The present disclosure also relates to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The disclosed system and method can also be embodied as computer readable code on a computer readable medium. The computer readable medium is any data storage device that can store data, which can thereafter be read by a computer system. Examples of the computer readable medium include hard drives, read-only memory, random-access memory, CD-ROMs, CD-Rs, CD-RWs, magnetic tapes and other optical and non-optical data storage devices. The computer readable medium can also be distributed over a network-coupled computer system so that the computer readable code is stored and executed in a distributed fashion.

The foregoing description has been directed to particular embodiments of the present disclosure. It will be apparent, however, that other variations and modifications may be made to the described embodiments, with the attainment of some or all of their advantages. The procedures, processes and/or modules described herein may be implemented in hardware, software, embodied as a computer-readable medium having program instructions, firmware, or a combination thereof. For example, the functions described herein may be performed by a processor executing program instructions out of a memory or other storage device. Therefore, it is the object of the appended claims to cover all such variations and modifications as come within the true spirit and scope of the present disclosure.

What is claimed is:

1. A system for implementing a modular workstation architecture for managing a medical analysis instrument, comprising:
a processor coupled to a memory unit and operable to retrieve and execute instructions stored in the memory unit to implement:
a data manager, an instrument manager and a user interface shell, each being communicatively coupled with each other via a communication interface;
the data manager being operative to host workstation services that are independent of specifications for the instrument, the services being arranged in a modular configuration and being communicatively coupled to a service manager for managing transactions involving the services;
the instrument manager being operative to host instrument specific functions and provide requests and responses to the data manager to fulfill service transactions; and
the user interface shell including components to provide input and output for a user and provide requests or responses to the data manager and the instrument manager to fulfill input or output transactions.

2. The system according to claim 1, further comprising:
another instrument manager being operative to host instrument specific functions for another instrument and provide requests and responses to the data manager to fulfill service transactions; and
the requests and responses of the instrument managers being in the form of application programming interface calls.

3. The system according to claim 1, wherein one or more of the data manager or the instrument manager further comprise a service discovery component for locating a service indicated by a request or a response originating from the data manager, the instrument manager or the user interface shell.

4. The system according to claim 1, wherein one or more of the data manager or the instrument manager further comprise a service configuration component for configuring services in the respective data manager or instrument manager.

5. The system according to claim 4, further comprising an XML file associated with a service configuration, the XML file being accessible by the service configuration component to determine a service configuration when the service is invoked.

6. The system according to claim 5, further comprising a configuration management tool that is accessible through a user interface that is available to one or more of the data manager, the instrument manager or the user interface shell, the configuration management tool being operative to create or modify XML code in the XML file to modify the service configuration.

7. The system according to claim 6, further comprising a plug-and-play service that can be implemented by installation through the configuration management tool.

8. The system according to claim 3, wherein the service that is locatable by the service discovery component includes one or more of a communication driver, a message handler or a security manager.

9. The system according to claim 1, further comprising an XML file associated with a component of the user interface shell, the XML file containing XML code for defining component behavior.

10. The system according to claim 1, wherein the data manager further comprises a localization manager for maintaining settings related to a locale in which the system is implemented, the localization manager being operative to permit selection of a locale related setting to implement a locale change while maintaining a loaded state for the user interface shell.

11. The system according to claim 1, further comprising a user interface shell tool for developing user interface shell components, the tool being operative to provide a common base of components such that a resulting user interface has a similar look and behavior to other user interfaces that are developed using the tool.

12. The system according to claim 1, wherein one or more of the data manager, instrument manager or user interface shell further comprises a security manager for authenticating users, the security manager being operative to receive authentication requests and return a security component representative of security authorization.

13. The system according to claim 12, wherein the data manager and instrument manager each include the security manager, and at least one security manager submits an authentication request to and receives a security component from the other security manager to authenticate a user.

14. The system according to claim 1, wherein the user interface shell further comprises a securable entity that is operative to expose security properties.

15. The system according to claim 14, wherein the security properties are configurable according to an XML file.

16. The system according to claim 15, wherein the data manager hosts user security data, and the securable entity includes features that are accessible by a user according to the security properties and a security level associated with the user in the user security data.

17. A method for implementing a modular workstation architecture on a numerical computation device for managing a medical analysis instrument, comprising:
hosting workstation services in a data manager that are independent of specifications for the instrument and arranging the services in a modular configuration;
hosting instrument specific functions in an instrument manager and providing requests and responses to the data manager to fulfill service transactions; and
providing components in a user interface shell to permit user input and output and to permit requests or responses to be applied to the data manager and the instrument manager to fulfill input or output transactions.

18. The method according to claim 17, further comprising locating a service indicated by a request or a response originating from the data manager, the instrument manager or the user interface shell.

19. The method according to claim 17, further comprising configuring services in the respective data manager or instrument manager using a service configuration component.

20. The method according to claim 19, further comprising accessing an XML file associated with a service configuration to determine a service configuration when the service is invoked.

21. The method according to claim 20, further comprising manipulating XML code in the XML file to modify the service configuration.

22. The method according to claim 18, wherein locating the service further comprises locating one or more of a communication driver, a message handler or a security manager.

23. The method according to claim 17, further comprising providing an XML file associated with a component of the user interface shell for defining component behavior.

24. The method according to claim 17, further comprising selecting a locale related setting to implement a locale change while maintaining a loaded state for the user interface shell.

25. The method according to claim 17, further comprising:
providing a user interface shell tool for developing user interface shell components to provide a common base of components; and
generating a user interface that provides a similar look and behavior to other user interfaces that are generated using the tool.

26. The method according to claim 17, further comprising authenticating users by receiving an authentication request and returning a security component representative of security authorization.

27. The method according to claim 17, further comprising assigning a characteristic to a component that is usable in the user interface shell to expose security properties.

28. The method according to claim 27, further comprising defining the security properties using XML code.

29. The method according to claim 28, further comprising accessing user security data and determining security access for the component according to the user security data and the security properties.

30. A system for implementing a modular workstation architecture for managing a medical analysis instrument, comprising:
a processor coupled to a memory unit and operable to retrieve and execute instructions stored in the memory unit to implement a data manager comprising;
a service manager coupled to a plurality of service components and operative to manage service transactions among the service components;
an object request broker for locating and loading service components; and
a message processor for processing messages between service components and being operative to pass messages to the object request broker to cause a service component to be located and loaded upon invocation of the service component;
wherein one of the service components is a configuration manager service for determining configuration settings for at least one service component, the configuration manager service being operative to provide configuration settings to the message processor when a service component is invoked, whereby the object request broker can load the service component in accordance with the configuration settings.
